(19) United States
(12) Patent Application Publication
(10) Pub. No.: US 2006/0171932 A1
(43) Pub. Date: Aug. 3, 2006

(54) Adipose derived stromal cells exhibiting characteristics of endothelial cells

(75) Inventors: James K. Hendricks, Elkridge, MD (US); James B. Mitchell II, Abingdon, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Cognate Therapeutics, Inc.

(21) Appl. No.: 11/342,096

(22) Filed: Jan. 27, 2006

Related U.S. Application Data
(60) Provisional application No. 60/648,630, filed on Jan. 31, 2005.

(51) Int. Cl. C12N 5/08; A61K 35/12

(52) U.S. Cl. 424/93.7; 435/366

(57) ABSTRACT

The present invention encompasses an adipose-derived adult stromal (ADAS) cell exhibiting at least one characteristic of a pre-endothelial cell and/or an endothelial cell. The present invention also encompasses compositions and methods for generating an adipose-derived adult stromal to exhibit at least one characteristic of a pre-endothelial cell and/or an endothelial cell. Methods for using the cells in vascular transplantation, tissue engineering, regulation of angiogenesis, vasculogenesis, and the treatment of numerous disorders including heart disease are also included.

### CROSS-REFERENCE TO RELATED APPLICATIONS

This application claims priority under 35 U.S.C. § 119(e) to U.S. Provisional Patent Application No. 60/648,630, filed Jan. 31, 2005, which is incorporated by reference herein in its entirety.

### BACKGROUND OF THE INVENTION

The neonatal period in human development is characterized by the presence of “stem” cells with the potential to develop along multiple differentiation pathways. The terminal differentiation of these cells is determined by cytokine and hormonal cues which co-ordinate organogenesis and tissue architecture. Embryonic stem (ES) cells from mice have been isolated and studied extensively in vitro and in vivo. Using exogenous stimuli in vitro, investigators have induced ES cell differentiation along multiple lineage pathways. These pathways include neuronal, B lineage lymphoid, and adipocytic (Dani, et al., 1997, J. Cell Sci. 110:1279; Remoncourt, et al., 1998, Mech. Dev. 79:185; O'Shea, 1999, Anat. Rec. 257:32).

Multipotential stem cells also exist in tissues of the adult organism. The best characterized example of an adult stem cell is the hematopoietic progenitor cell isolated from the bone marrow and peripheral blood. In the absence of treatment, lethally irradiated mice died because they failed to replenish their circulating blood cells; however, transplantation of bone marrow cells from syngeneic donor animals rescued the host animal. The donor cells were responsible for repopulating the circulating blood cells. Studies have since been conducted to demonstrate that undifferentiated hematopoietic stem cells are capable of regenerating the different blood cell lineages in a host animal. These studies have provided the basis for bone marrow transplantation, a widely accepted therapeutic modality for cancer and inborn errors of metabolism.

Bone marrow derived cells have also been found to be capable of differentiating into other cell types. Bone marrow contains at least two types of stem cells, hematopoietic stem cells and stem cells of non-hematopoietic tissues variously referred to as mesenchymal stem cells or marrow stromal cells (MSCs) or bone marrow stromal cells (BMSCs). These terms are used synonymously throughout herein. MSCs are of interest because they are easily isolated from an aspirate of bone marrow and they readily generate single-cell derived colonies. The single-cell derived colonies can be expanded through as many as 50 population doublings in about 10 weeks, and can differentiate into osteoblasts, adipocytes, chondrocytes (Friedenstein, et al., 1970, Cell Tissue Kinet. 3:393-403; Castro-Malaspina, et al., 1980, Blood 56:289-301; Beresford, et al., 1992, J. Cell Sci. 102:341-351; Prockop, 1997, Science 276:71-74), myocytes (Wakitani, et al., 1995, Muscle Nerve 18:1417-1426), astrocytes, oligodendrocytes, and neurons (Azizi, et al., 1998, Proc. Natl. Acad. Sci. USA 95:3908-3913; Kopen, et al., 1999, Proc. Natl. Acad. Sci. USA 96:10711-10716; Chopp, et al., 2000, Neuroreport II, 3001-3005; Woodbury, et al., 2000, Neuroscience Res. 61:364-370).

Furthermore, MSCs give rise to cells of all three germ layers (Kopen, et al., 1999, Proc. Natl. Acad. Sci. 96:10711-10716; Liechty, et al., 2000, Nature Med. 6:1282-1286; Kottonet, et al., 2001, Development 128:5181-5188; Toma, et al., 2002, Circulation 105:93-98; Jiang, et al., 2002, Nature 418:41-49). In vivo evidence indicates that unfractionated bone marrow-derived cells as well as pure populations of MSCs give rise to epithelial cell-types including those of the lung (Krause, et al., 2001, Cell 105:369-377; Petersen, et al., 1999, Science 284:1168-1170) and several recent studies have shown that engraftment of MSCs is enhanced by tissue injury (Ferrari, et al., 1998, Science 279:1528-1530; Okamoto et al., 2002, Nature Med. 8:1101-1017). For these reasons, MSCs are currently being tested for their potential use in cell and gene therapy of a number of human diseases (Horwitz, et al., 1999, Nat. Med. 5:309-313; Caplan, et al., 2000, Clin. Orthoped. 379:567-570).

MSCs constitute an alternative source of pluripotent stem cells. Under physiological conditions they maintain the architecture of bone marrow and regulate hematopoiesis with the help of different cell adhesion molecules and the secretion of cytokines, respectively (Clark, et al., 1995, Ann. NY Acad. Sci. 770:70-78). MSCs grown out of bone marrow by their selective attachment to tissue culture plastic can be efficiently expanded (Azizi, et al., 1998, Proc. Natl. Acad. Sci. USA 95:3908-3913; Colter, et al., 2000, Proc. Natl. Acad. Sci. USA 97:3213-218) and genetically manipulated (Schwarz, et al., 1999, Hum. Gene Ther. 10:2539-2549).

MSCs are also referred to as mesenchymal stem cells because they are capable of differentiating into multiple mesodermal tissues, including bone (Beresford, et al., 1992, J. Cell Sci. 102:341-351), cartilage (Lennon, et al., 1995, Exp. Cell Res. 219:211-222), fat (Beresford, et al., 1992, J. Cell Sci. 102:341-351) and muscle (Wakitani, et al., 1995, Muscle Nerve 18:1417-1426). In addition, differentiation into neuron-like cells expressing neuronal markers has been reported (Woodbury, et al., 2000, J. Neurosci. Res. 61:364-370; Sanchez-Ramos, et al., 2000, Exp. Neurol. 164:247-256; Deng, et al., 2001, Biochem. Biophys. Res. Commun. 282:148-152), suggesting that MSC may be capable of overcoming germ layer commitment. Based on these findings, the bone marrow has been proposed as a source of stromal stem cells for regeneration of bone, cartilage, muscle, adipose tissue, liver, neuronal, and other tissues. However, extraction of bone marrow stromal cells presents a high level of risk and discomfort to the donor.

In contrast, adult human extramedullary adipose tissue-derived stromal cells (ADAS) represent a stromal stem cell source that can be harvested routinely with minimal risk or discomfort to the patient. Pathologic evidence suggests that adipose-derived stromal cells are capable of differentiation along multiple lineage pathways. Furthermore, it has been demonstrated that stromal cells from adipose tissue are capable of differentiating into multiple mesodermal tissues.

Vasculogenesis, the in situ differentiation of the primitive endothelial progenitors known as angioblasts into endothelial cells that aggregate into a primary capillary plexus, is responsible for the development of the vascular system during embryogenesis (Peichev, et al., 2000, Blood 95:952-958). In contrast, angiogenesis, defined as the formation of new blood vessels by a process of sprouting from preexisting vessels, occurs both during development and in postnatal life (Peichev, et al., 2000, Blood 95:952-958; Watt, et al., 1995, Leuk. Lymphoma 17:229-235; Reyes, et al., 2001, Blood 98:2615-2625). Until recently, it was thought that blood vessel formation in postnatal life was mediated by sprouting of endothelial cells from existing vessels. However, recent studies have suggested that endothelial stem cells may persist into adult life, where they contribute to the formation of new blood vessels (Nishikawa, et al., 1998, Development 125:1747-1757; Gehling, et al., 2000, Blood 95:3106-3112; Rafii, et al., 1994, Blood 84:10-18; Asahara, et al., 1997, Science 275:964-967). This in turn suggests that, as during development, neoangiogenesis in the adult may depend at least in part on a process of vasculogenesis. Precursors of endothelial cells have been isolated from bone marrow and peripheral blood (Peichev, et al., 2000, Blood 95:952-958; Watt, et al., 1995, Leuk. Lymphoma 17:229-235). The ontogeny of these endothelial progenitors is unknown.

Therefore, methods for the isolation and propagation of an easily obtainable source of progenitor cells that can give rise to endothelial cells are needed. Current methods for culturing and obtaining a large number of endothelial progenitor cells have been unsuccessful. The availability of a large number of endothelial progenitor cells would be extremely useful in vascular transplantation, tissue engineering, regulation of angiogenesis, vasculogenesis, and the treatment of numerous disorders including heart disease.

Thus, there is a long felt need for methods and compositions for standardizing culture conditions for maximizing the proliferation of endothelial progenitor cells for obtaining large number of such cells useful for therapeutic and experimental purposes. The present invention satisfies this need.

### BRIEF SUMMARY OF THE INVENTION

The present invention includes compositions and methods for generating an adipose-derived adult stromal (ADAS) cell exhibiting at least one characteristic of a pre-endothelial cell and/or an endothelial cell.

In one aspect, the ADAS cell is induced to differentiate in vitro.

In another aspect, the ADAS cells is induced to differentiate in vivo.

In a further aspect, the ADAS cell has been engineered to express exogenous genetic material.

In yet another aspect, the ADAS cells is derived from a human.

The invention also includes an ADAS cell induced to expresses at least one of CD34 and CD31.

In one aspect, the ADAS cell expresses at least one of CD34 and CD31 at a higher level when compared with the expression level of CD34 and CD31, respectively, from an otherwise identical ADAS cell not induced to express at least one characteristic of a pre-endothelial cell.

In another aspect, the ADAS cell expresses at least one of CD34, CD31, CD40, CD63, or a combination thereof.

In yet another aspect, the ADAS cell expresses at least one of CD34, CD31, CD40, CD63, or a combination thereof at a higher level when compared with the expression level of CD34, CD31, CD40 and CD63, respectively, from an otherwise identical ADAS cell not induced to express at least one characteristic of a pre-endothelial cell.

The present invention also includes a method of differentiating an ADAS cell to express at least one characteristic of a pre-endothelial cell, the method comprising incubating said cell in MII medium followed by incubating said cell in MIII medium.

In one aspect, the method includes using an ADAS cell derived from a human.

In another aspect, MII medium comprises N2 supplement, B27 supplement, glutamine and fibroblast growth factor (FGF).

In yet another aspect, the concentration of glutamine in MII medium is about 2.3 mM.

In a further aspect, the concentration of FGF in MII medium is about 10 ng/mL.

In one aspect, MIII medium comprises N2 supplement, B27 supplement, glutamine, nicotinamide and fetal bovine serum (FBS).

In another aspect, the concentration of glutamine in MIII medium is about 2.3 mM.

In yet another aspect, the concentration of nicotinamide in MIII medium is about 10 mM.

In a further aspect, the concentration of FBS in MIII medium is about 2%.

The invention also includes a method of inducing vasculogenesis in an animal, the method comprising a) inducing an isolated adipose tissue-derived adult stromal (ADAS) cell to express at least one characteristic of a pre-endothelial cell; and b) administering said cell so induced into said animal.

In one aspect, the ADAS cell is autologous to the animal.

In another aspect, the ADAS cell is isolated from an allogeneic donor.

In a further aspect, the ADAS cell is isolated from a xenogeneic donor.

In yet another aspect, the ADAS cell is derived from a human.

The invention also includes a method of determining the ability of a compound to affect the differentiation of an ADAS cell into a pre-endothelial cell and/or endothelial cell, the method comprising:
a) culturing said ADAS cell in a stromal cell medium for a period of time;
b) replacing said stromal cell medium with a differentiation medium comprising a compound or a control vehicle;
c) incubating said ADAS cell in said differentiation medium comprising said compound or said control vehicle for a period of time;
d) determining the number or percentage of differentiated cells using said differentiation medium comprising said compound from step (c);
e) determining the number of percentage of differentiated cells in the cells using said differentiation medium containing said vehicle alone from step (c);
f) comparing the number or percentage of differentiated cells from steps (d) and (e);
g) a greater number of percentage of differentiated cells from step (d) compared to the number of percentage of differentiated cells from step (e) indicates that said compound is capable of inducing differentiation of said ADAS cell into a pre-endothelial cell and/or endothelial cell.

In one aspect, the ADAS cell is derived from a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through IF, is a series of images depicting ADAS cultures untreated and treated with MII/MIII. FIGS. 1A and 1B depict pre-treated and untreated control ADAS cultures, respectively. FIGS. 1C and 1D depict ADAS cultures treated with MII. FIGS. 1E and 1F depict ADAS cultures treated with MIII.

### DETAILED DESCRIPTION OF THE INVENTION

The invention provides compositions and methods for inducing adipose tissue-derived adult stromal (ADAS) cells to express at least one characteristic of a pre- endothelial cell and/or an endothelial cell. The cells produced by the methods of this invention provide a source of functional cells that can be used for research, transplantation, and development of tissue engineering products for the treatment of diseases and tissue repair.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “adipose derived stromal cells,” “adipose tissue-derived stromal cells,” or “adipose tissue-derived adult stromal (ADAS) cells” are used interchangeably and refer to stromal cells that originate from adipose tissue which can serve as stem cell-like precursors to a variety of different cell types such as osteocytes, chondrocytes, and adipocytes.

“Adipose” refers to any fat tissue. The adipose tissue may be brown or white adipose tissue. Preferably, the adipose is subcutaneous white adipose tissue. Such cells may comprise a primary cell culture or an immortalized cell line. The adipose tissue may be from any organism having fat tissue. Preferably the adipose tissue is mammalian, most preferably the adipose tissue is human. A convenient source of human adipose tissue is from liposuction surgery. However, the source of adipose tissue or the method of isolation of adipose tissue is not critical to the invention.

“Allogeneic” refers to a graft derived from a different animal of the same species.

As defined herein, an “allogeneic adipose derived adult stromal cell” is obtained from a different individual of the same species as the recipient.

“Alloantigen” is an antigen that differs from an antigen expressed by the recipient.

“Donor antigen” refers to an antigen expressed by the donor tissue to be transplanted into the recipient.

As used herein, an “effector cell” refers to a cell which mediates an immune response against an antigen. In the situation where a transplant is introduced into a recipient, the effector cells can be the recipient's own cells that elicit an immune response against an antigen present in the donor transplant. In another situation, the effector cell can be part of the transplant, whereby the introduction of the transplant into a recipient results in the effector cells present in the transplant eliciting an immune response against the recipient of the transplant.

As used herein, the term “autologous” is meant to refer to any material derived from the same individual to which it is later to be re-introduced into the individual.

As used herein, the term “angiogenesis” refers to the process by which new blood vessels are generated from existing vasculature and tissue (Folkman, 1995, Nat. Med. 1:37-31). The phrase “repair or remodeling” refers to the reformation of existing vasculature. The alleviation of tissue ischemia is critically dependent upon angiogenesis. The spontaneous growth of new blood vessels provides collateral circulation in and around an ischemic area, improves blood flow, and alleviates the symptoms caused by the ischemia.

As used herein, the term “angiogenic factor” or “angiogenic protein” refers to any known protein capable of promoting growth of new blood vessels from existing vasculature (“angiogenesis”). Suitable angiogenic factors for use in the invention include, but are not limited to, placenta growth factor, macrophage colony stimulating factor (M-CSF), granulocyte macrophage colony stimulating factor (GM-CSF), vascular endothelial growth factor (VEGF)-A, VEGF-B, VEGF-C, VEGF-D, VEGF-E, neuropilin, fibroblast growth factor (FGF)-1, FGF-2 (bFGF), FGF-3, FGF-4, FGF-5, FGF-6, Angiopoietin 1, Angiopoietin 2, erythropoietin (EPO), bone morphogenic protein (BMP)-2, BMP-4, BMP-7, TGF-β, IGF-1, Osteopontin, Pleiotropin, Activin, Endothelin-1 and combinations thereof. Angiogenic factors can act independently, or in combination with one another. When in combination, angiogenic factors can also act synergistically, whereby the combined effect of the factors is greater than the sum of the effects of the individual factors taken separately. The term “angiogenic factor” or “angiogenic protein” also encompasses functional analogues of such factors. Functional analogues include, for example, functional portions of the factors. Functional analogues also include anti-idiotypic antibodies which bind to the receptors of the factors and thus mimic the activity of the factors in promoting angiogenesis and/or tissue remodeling. Methods for generating such anti-idiotypic antibodies are well known in the art and are described, for example, in WO 97/23510, the contents of which are incorporated by reference herein.

“Angiogenic factors” as used herein can be produced or obtained from any suitable source. For example, the factors can be purified from their native sources, or produced synthetically or by recombinant expression. The factors can be administered to patients as a protein composition. The factors can be administered in the form of an expression plasmid encoding the factors. The construction of suitable expression plasmids is well known in the art. Suitable vectors for constructing expression plasmids include, for example, adenoviral vectors, retroviral vectors, adeno-associated viral vectors, RNA vectors, liposomes, cationic lipids, lentiviral vectors and transposons.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiated associated proteins in a given cell. For example, expression of endothelial cell markers CD31 and von Willebrand factor and formation of a “cobblestone” morphology is a typical example of differentiated mature endothelial cells. When a cell is said to be “differentiated,” as that term is used herein, the cell is in the process of being differentiated.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

An “endothelial ADAS cell” is used herein to refer to an ADAS cell expressing at least one characteristic of a pre-endothelial cell and/or an endothelial cell.

“Expandability” is used herein to refer to the capacity of a cell to proliferate, for example, to expand in number or in the case of a cell population to undergo population doublings.

“Graft” refers to a cell, tissue, organ or otherwise any biological compatible lattice for transplantation.

By “growth factors” is intended the following specific factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

As used herein, the term “multipotential” or “multipotentiality” is meant to refer to the capability of a stem cell of the central nervous system to differentiate into more than one type of cell.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into, for example, endothelial cells or endothelial-like cells; or a lineage-committed progenitor cell and its progeny, which is capable of self-renewal and is capable of differentiating into an endothelial cell or endothelial-like cell. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

The term “pre-endothelial cell” refers to a cell which is potentially capable of an unlimited number of mitotic divisions to either renew itself or to produce progeny cells which will differentiate into endothelial cells or endothelial-like cells.

The term “stromal cell medium” as used herein refers to a medium useful for culturing ADAS cells. Typically, the stromal cell medium comprising a base medium, serum and an antibiotic/antimycotic. However, ADAS cells can be cultured with stromal cell medium without an antibiotic/antimycotic and supplemented with at least one growth factor. Preferably the growth factor is human epidermal growth factor (hEGF). The preferred concentration of hEGF is about 1-50 ng/ml, more preferably the concentration is about 5 ng/ml. The preferred base medium is DMEM/F12 (1:1). The preferred serum is fetal bovine serum (FBS) but other serum may be used including fetal calf serum (FCS), horse serum or human serum. Preferably up to 20% FBS will be added to the above media in order to support the growth of stromal cells. However, a defined medium could be used if the necessary growth factors, cytokines, and hormones in FBS for stromal cell growth are identified and provided at appropriate concentrations in the growth medium. It is further recognized that additional components may be added to the culture medium. Such components include but are not limited to antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing stromal cells. Rather, any media capable of supporting stromal cells in tissue culture may be used.

“MII/MIII medium regimen” refers to the incubation of ADAS cells with MII medium followed by the incubation of the cells with MIII medium.

“Transplant” refers to a biocompatible lattice or a donor tissue, organ or cell, to be transplanted.

As used herein, a “therapeutically effective amount” is the amount of ADAS cells expressing at least one characteristic of a pre-endothelial cell and/or an endothelial cell which is sufficient to provide a beneficial effect to the subject to which the cells are administered.

“Xenogeneic” refers to a graft derived from an animal of a different species.

As used herein “endogenous” refers to any material from or produced inside an organism, cell or system.

“Exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence(s).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Description

Adipose tissue offers an alternative to bone marrow as a source of stem cells. Adipose tissue is readily accessible and abundant in many individuals. Stem cells derived from adipose tissue can be harvested by liposuction which is a relatively non-invasive procedure and can yield an abundant quantity of adipose-derived adult stromal (ADAS) cells.

The present invention relates to the discovery that ADAS cells can be treated with a defined culture medium to express at least one characteristic of a pre-endothelial cell and/or an endothelial cell. These cells are referred to herein as “endothelial ADAS cells.” Therefore, based upon the disclosure herein, a large population of endothelial ADAS cells expressing at least one characteristic of a pre-endothelial cell and/or an endothelial cell can be generated and expanded while preserving their ability to differentiate into mature endothelial cells. As such, the present invention comprises compositions and methods for generating large numbers of endothelial ADAS cells useful for experimental/therapeutic purposes.

I. Isolation and Culturing of ADAS

The ADAS cells useful in the methods of the present invention may be isolated by a variety of methods known to those skilled in the art. For example, such methods are described in U.S. Pat. No. 6,153,432, which is incorporated herein in its entirety. In a preferred method, ADAS cells are isolated from a mammalian subject, preferably a human subject. In humans, the ADAS cells are typically isolated from liposuction material. If the cells of the invention are to be transplanted into a human subject, it is preferable that the ADAS cells be isolated from that same subject so as to provide for an autologous transplant.

In another aspect of the invention, the administered ADAS cells may be allogeneic with respect to the recipient. The allogeneic ADAS cells are isolated from a donor that is a different individual of the same species as the recipient. Following isolation, the cells are cultured using the methods disclosed herein to produce an allogeneic product. The invention also encompasses ADAS cells that are xenogeneic with respect to the recipient.

Without limiting the invention in anyway, stromal cells from adipose tissue can be isolated using the methods disclosed herein. Briefly, human adipose tissue from subcutaneous depots are removed by liposuction surgery. The adipose tissue is then transferred from the liposuction cup into a 500 ml sterile beaker and allowed to settle for about 10 minutes. Precipitated blood is removed by suction. About a 125 ml volume (or less) of the tissue is transferred to a 250 ml centrifuge tube, and the tube is then filled with Krebs-Ringer Buffer. The tissue and buffer are allowed to settle for about three minutes or until a clear separation is achieved, and then the buffer is removed by aspiration. The tissue can be washed with Krebs-Ringer Buffer for an additional four to five times or until the tissue becomes orange-yellow in color and until the buffer becomes light tan in color.

The stromal cell of the adipose tissue can be dissociated using collagenase treatment. Briefly, the buffer is removed from the tissue and replaced with about 2 mg collagenase/ml Krebs Buffer (Worthington, ME) solution at a ratio of 1 ml collagenase solution/ml tissue. The tubes are incubated in a 37° C. water bath with intermittent shaking for about 30 to 35 minutes.

Stromal cells are isolated from other components of the adipose tissue by centrifugation for 5 minutes at 500×g at room temperature. The oil and adipocyte layer are removed by aspiration. The remaining fraction can be resuspended in approximately 100 ml of phosphate buffered saline (PBS) by vigorous swirling, divided into 50 ml tubes and centrifuged for five minutes at 500×g. The buffer is removed by aspiration, leaving the stromal cells. The stromal cells are then resuspended in stromal cell medium, and plated at an appropriate cell density and incubated at 37° C. in 5% CO₂ overnight. Once attached to the tissue culture dish or flask, the cultured stromal cells can be used immediately or maintained in culture for a period of time or a number of passages before being induced to differentiate into the desired cell, for example cells expressing at least one characteristic of a pre-endothelial cell and/or an endothelial cell as described in the Example section. However, the invention should in no way be construed to be limited to any one method of isolating stromal cells. Rather, any method of isolating ADAS cells should be encompassed in the present invention.

Any medium capable of supporting fibroblasts in cell culture may be used to culture ADAS. Media formulations that support the growth of fibroblasts include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), and the like. A preferred medium for culturing ADAS is DMEM, more preferably DMEM/F12 (1:1).

Additional non-limiting examples of media useful in the methods of the invention can contain fetal serum of bovine or other species at a concentration at least 1% to about 30%, preferably at least about 5% to 15%, most preferably about 10%. Embryonic extract of chicken or other species can be present at a concentration of about 1% to 30%, preferably at least about 5% to 15%, most preferably about 10%.

Following isolation, ADAS cells are incubated in stromal cell medium in a culture apparatus for a period of time or until the cells reach confluency before passing the cells to another culture apparatus. The culturing apparatus can be of any culture apparatus commonly used in culturing cells in vitro. A preferred culture apparatus is a culture flask with a more preferred culture apparatus being a T-225 culture flask. ADAS cells can be cultured with stromal cell medium without an antibiotic/antimycotic and supplemented with at least one growth factor. Preferably the growth factor is human epidermal growth factor (hEGF). The preferred concentration of HEGF is about 1-50 ng/ml, more preferably the concentration is about 5 ng/ml.

ADAS cells can be cultured in stromal cell medium supplemented with hEGF in the absence of an antibiotic/antimycotic for a period of time or until the cells reach a certain level of confluence. Preferably, the level of confluence is greater than 70%. More preferably, the level of confluence is greater than 90%. A period of time can be any time suitable for the culture of cells in vitro. Stromal cell medium may be replaced during the culture of the ADAS cells at any time. Preferably, the stromal cell medium is replaced every 3 to 4 days. ADAS cells are then harvested from the culture apparatus whereupon the ADAS cells can be used immediately or cryopreserved to be stored for use at a later time. ADAS cells may be harvested by trypsinization, EDTA treatment, or any other procedure used to harvest cells from a culture apparatus.

II. Treatment of ADAS Cells

The invention comprises the treatment of the ADAS cells to induce them to express at least one characteristic of a pre-endothelial and/or an endothelial cell (these cells are referred as endothelial ADAS cells). While not wishing to be bound by any particular theory, it is believed that the treatment of the ADAS cells with a defined medium containing a combination of serum, embryonic extracts, preferably a non-human embryonic extract, purified or recombinant growth factors, cytokines, hormones, and/or chemical agents, in a 2-dimensional or 3-dimensional biocompatible lattice, induces the ADAS cell to differentiate.

MII Medium:

Freshly isolated or cryopreserved ADAS cells can be used for the following treatment with a differentiation medium in order to induce the ADAS cells to exhibit at least one characteristic of a pre-endothelial and/or an endothelial cell. Untreated ADAS cells are cultured in any growth medium, for example a medium comprising DMEM/F12 (1:1), 10% FBS, 5 ng/mL hEGF and 1 ng/mL hFGF in order to compare the effects of treating an otherwise identical ADAS cell with a differentiation medium. For example, the ADAS cells in the treatment group can be cultured with MII medium which comprises DMEM/F12, N2 supplement, B27 supplement, glutamine and FGF. In one embodiment of the present invention, MII medium does not contain serum.

Preferably, the concentration of glutamine in MII medium is at least 0.5 mM to about 25 mM, preferably at least about 1 mM to 20 mM, more preferably at least about 1.5 mM to 15 mM, even more preferably at least about 1.5 mM to 10 mM, most preferably at least about 2 mM to 5 mM. In one aspect of the present invention, the concentration of glutamine is about 2.3 mM.

The concentration of hFGF in MII medium is at least 0.5 ng/mL to about 100 ng/mL, preferably at least about 1 ng/mL to 75 ng/mL, more preferably at least about 1.5 ng/mL to 50 ng/mL, even more preferably at least about 2 ng/mL to 25 ng/mL, most preferably at least about 3 ng/mL to 15 ng/mL. In one aspect of the present invention, the concentration of hFGF in MII medium is about 10 ng/mL.

The ADAS cells can be treated with MII medium for a period of time sufficient to change the phenotype/morphology of the ADAS to exhibit at least one characteristic of a pre-endothelial and/or an endothelial cell. Preferably, the ADAS cells are subjected to a stepwise treatment regimen beginning with an initial treatment of MII medium for about 6 days, with medium changes of MII medium on days 1, 3 and 5 following the initial plating. Based on the present disclosure, one skilled in the art would appreciate that the ADAS cells can be treated with MII medium for more than 6 days, for example the ADAS cells can be treated for about one week, two weeks, one month, two months, or even 6 months; and the MII medium can be changed at anytime during the treatment duration.

ADAS cells are incubated in MII medium for a period of time or until the cells reach a certain level of confluence. Preferably the level of confluence is greater than 70%. More preferably the level of confluence is greater than 90%. The period of time in which the cells are cultured in MII medium can be any time suitable for the culturing of cells in vitro.

Without wishing to be bound by any particular theory, it is believed that the treatment of ADAS cells with MII medium alters the phenotype and morphology of the ADAS cells. For example, when compared to untreated or pre-treated ADAS cells, ADAS cells treated with MII medium exhibit a less fibroblastic morphology and are rounder in shape forming a network of cell-to-cell connections.

Following the treatment of the ADAS cells with MII medium, the ADAS cells can be harvested for experimental/therapeutic use immediately or cryopreserved to be used at a later time. In one aspect of the present invention, the MII medium treated ADAS cells are further treated with MIII medium as more fully discussed below.

MIII Medium:

Following the treatment of ADAS cells with MII medium, the cells can further be treated with MIII medium which comprises DMEM/F12, N2 supplement, B27 supplement, glutamine, nicotinamide, FBS. The treatment of ADAS cells with MII medium following by MIII is also referred as MII/MIII medium. Without wishing to be bound by any particular theory, it is believed that the treatment of ADAS cells with MIII medium following the treatment of the ADAS with MII medium further differentiates the cells towards the endothelial lineage. The treatment of ADAS cells with MIII medium typically follows the MII treatment and a washing step using PBS. The washing step using PBS serves to remove components of the MII medium from the cell culture prior to culturing ADAS cells with MIII medium. However, the invention should not be limited to treating the ADAS cells with MIII medium following the treatment of the ADAS with MII medium. The invention should encompass using MIII medium at any time to differentiate the ADAS cells towards the endothelial lineage.

Preferably, the concentration of glutamine in MIII medium is at least 0.5 mM to about 25 mM, preferably at least about 1 mM to 20 mM, more preferably at least about 1.5 mM to 15 mM, even more preferably at least about 1.5 mM to 10 mM, most preferably at least about 2 mM to 5 mM. In one aspect of the present invention, the concentration of glutamine is about 2.3 mM.

The concentration of nicotinamide in MIII medium is at least 0.5 mM to about 100 mM, preferably at least about 1 mM to 75 mM, more preferably at least about 1.5 mM to 50 mM, even more preferably at least about 2 mM to 25 mM, most preferably at least about 3 mM to 15 mM. In one aspect of the present invention, the concentration of nicotinamide in MIII medium is about 10 mM.

The concentration of FBS in MIII medium is at least 0.5% to about 20%, preferably at least about 0.75% to 15%, more preferably at least about 1% to 10%, even more preferably at least about 1.5% to 7.5%, most preferably at least about 1.75% to 5%. In one aspect of the present invention, the concentration of FBS in MIII medium is about 2%.

The ADAS cells can be treated with MIII medium for a period of time sufficient to change the phenotype of each cell type to exhibit at least one characteristic of a pre-endothelial and/or an endothelial cell. Preferably, the ADAS cells are treated with MIII medium for about 4 days. Based on the present disclosure, one skilled in the art would appreciate that the cells can be treated with MIII medium for any periods of time. For example the cells can be treated with MIII medium for more than 4 days (i.e. the cells can be treated for about one week, two weeks, one month, two months, or even 6 months). Further, the cells can be treated with MIII medium for less than 4 days (i.e. the cells can be treated for about 1 day, 2 days, or even 3 days). The MIII medium can be changed at anytime during the treatment duration.

ADAS cells are incubated in MIII medium for a period of time or until the cells reach a certain level of confluence. Preferably the level of confluence is greater than 70%. More preferably the level of confluence is greater than 90%. The period of time in MIII medium can be any time suitable for the culture of cells in vitro.

The treatment of ADAS cells with MIII medium further alters the phenotype and morphology of the cells to exhibit at least one characteristic of a pre-endothelial and/or an endothelial cell. When compared to untreated/pre-treated ADAS cells, ADAS cells treated with MII medium followed by MIII medium exhibited a change in the overall morphology of the cultured cell, for example, yielding a heterogeneous mixture of cells resembling those observed during the MII treatment in addition to cells forming “cobblestone” type areas resembling cultured endothelial cells.

Characterization:

The cells of the present invention, at any time point during the treatment of the cells with the MII/MIII medium regimen, can be harvested via trypsinization and collected for immediate experimental/therapeutic use or cryopreserved for use at a later time. As discussed elsewhere herein, the MII/MIII medium regimen refers to the incubation of ADAS cells with MII medium followed by the incubation of the cells with MIII medium. In one aspect of the invention the cells are cryopreserved at any step during the culturing or treatment regimen of the ADAS cells. Cryopreservation is a procedure common in the art and as used herein encompasses all procedures currently used to cryopreserve cells for future analysis and use. In another aspect, the cells can be harvested and subjected to flow cytometry to evaluate cell surface markers to assess the change in phenotype of the cells in view of the treatment regimen.

The ADAS cells and/or endothelial ADAS cells may be characterized in any one of numerous methods in the art and methods disclosed herein. The cells may be characterized by the identification of surface and intracellular proteins, genes, and/or other markers indicative of the differentiation of the cells to express at least one characteristic of a pre-endothelial cell and/or an endothelial cell. These methods will include, but are not limited to, (a) detection of cell surface proteins by immunofluorescent assays such as flow cytometry or in situ immunostaining of cell surface proteins such as CD80, CD86, CD14, CD45, CD34, CD133, CD90, CD105, HLA-DR, CD63, CD166, MHC Class I; CD44, CD73, CD54; CD31, CD13, CD40; CD29, CD49a, CD11, CD44, CD146; (b) detection of intracellular proteins by immunofluorescent methods such as flow cytometry or in situ immunostaining using specific monoclonal antibodies; (c) detection of the expression mRNAs by methods such as polymerase chain reaction, in situ hybridization, and/or other blot analysis.

Phenotypic markers of the desired cells are well known to those of ordinary skill in the art. Additional phenotypic markers continue to be disclosed or can be identified without undue experimentation. Any of these markers can be used to confirm that the ADAS cells exhibit at least one characteristic of a pre-endothelial cell and/or an endothelial cell. Lineage specific phenotypic characteristics can include cell surface proteins, cytoskeletal proteins, cell morphology, and secretory products.

Endothelial characteristics include the expression of endothelial markers such as CD29, CD31, CD34, CD54, CD61, CD 62E, CD105, CD144, CD184/CXC4, CD202b, and Mad-CAM-1. One of ordinary skill in the art would recognize upon the present disclosure that known calorimetric, fluorescent, immunochemical, polymerase chain reaction, chemical or radiochemical methods can readily ascertain the presence or absence of a pre-endothelial or an endothelial specific marker.

The present invention encompasses a cell population resulting from the incubation of ADAS cells according to the regimen disclosed herein. For example, the present invention includes a cell population comprising endothelial ADAS cells which have been cultured according to the MII/MIII medium regimen.

In one aspect of the invention, endothelial ADAS cells are at least positive for CD34 after culture in MII/MIII medium as measured by using the methods disclosed herein. In another aspect, endothelial ADAS cells express at least CD34 at a higher level when compared with the expression level of CD34 from an otherwise identical ADAS not cultured according to the MII/MIII medium regimen.

In another aspect of the invention, endothelial ADAS cells are at least positive for one of CD34 and CD31 following culture in MII/MIII medium. In a further aspect, endothelial ADAS cells express at least one of CD34 and CD31 at a higher level when compared with the expression level of CD34 and CD31, respectively, from an otherwise identical ADAS not cultured according to the MII/MIII medium regimen.

In an aspect of the invention, the endothelial ADAS cells are at least positive for one of CD34, CD31, CD40, CD63, or a combination thereof. In another aspect, endothelial ADAS cells express at least one of CD34, CD31, CD40, CD63, or a combination thereof at a higher level when compared with the expression level of CD34, CD31, CD40, CD63 or combination thereof, respectively, from an otherwise identical ADAS not cultured according to the MII/MIII medium regimen.

The present invention also provides methods for the identification and study of compounds that enhance differentiation of ADAS cells into cells expressing at least one characteristic of a pre-endothelial cell and/or an endothelial cell. Accordingly, a method is provided for determining the ability of a compound to affect the differentiation of an ADAS cell into an ADAS expressing at least one characteristic of a pre-endothelial cell and/or an endothelial cell comprising:
a) culturing an ADAS cell in a stromal cell medium for a period of time;
b) replacing the stromal cell medium with a differentiation medium comprising a compound or a control vehicle;
c) incubating the ADAS cell in the differentiation medium comprising the compound or the control vehicle for a period of time;
d) determining the number or percentage of differentiated cells using said differentiation medium comprising said compound from step (c);
e) determining the number of percentage of differentiated cells in the cells using said differentiation medium containing said vehicle alone from step (c);
f) comparing the number or percentage of differentiated cells from steps (d) and (e);
g) a greater number of percentage of differentiated cells from step (d) compared to the number of percentage of differentiated cells from step (e) indicates that said compound
is capable of inducing differentiation of said ADAS cell into an ADAS cell expressing at least one characteristic of a pre-endothelial cell and/or an endothelial cell.
   Methods of Using ADAS Cells

Following incubation of ADAS cells according to the MII/MIII medium regimen to induce the ADAS cells to express at least one characteristic of a pre-endothelial cell and/or an endothelial cell, the endothelial ADAS cells may be used to treat patients suffering from disorders or diseases associated with impairment in vasculogenesis and/or angiogenesis. The present invention includes compositions and methods in using endothelial ADAS cells for cell therapy to improve vasculogenesis and/or angiogenesis in a patient in need thereof. The endothelial ADAS cells produced according to the methods herein can be used to repair or replace damaged/destroyed endothelial tissue, to augment existing endothelial tissue, to introduce new or altered tissue, to modify artificial prostheses, or to join biological tissues or structures. For example, the cells can be used to replace heart valve cells. In addition, the cells can be used to treat ischemic myocardium following a myocardial infarction. Without wishing to be bound by any particular theory, it is believed that endothelial ADAS cells contribute to the regeneration of ischemic myocardium by modulating angiogenesis and myogenesis, cardiomyocyte apoptosis, and remodeling in the ischemic cardiac tissue.

The cells of the invention can further be used to treat cardiovascular diseases and disorders. The cells obtained by the methods of the present invention have several properties that can contribute to reducing and/or minimizing damage and promoting myocardial or cardiovascular repair and regeneration following damage. These include, but are not limited to, the ability to synthesize and secrete growth factors stimulating new blood vessel formation, the ability to synthesize and secrete angiogenic factors, the ability to synthesize and secrete growth factors stimulating cell survival and proliferation, the ability to proliferate and differentiate into cells directly participating in new blood vessel formation, and the ability to engraft damaged myocardium and inhibit scar formation (collagen deposition and cross-linking).

The cells of the invention can express numerous angiogenic growth factors, including but not limited to, placenta growth factor (PGF) and vascular endothelial growth factor (VEGF), which function in blood vessel formation and development of blood vessels, support ischemic tissue survival, induce reperfusion following occlusion/reperfusion injury of the hind limb, home to the heart when injected into animals after heart injury, and differentiate into cells expressing markers consistent with their differentiation into cells involved in vasculogenesis and angiogenesis. One skilled in the art would appreciate that the cells of the invention can incorporate into sites of angiogenesis after tissue ischemia for example in the limb, retina, and myocardium.

The present invention also includes methods for treating a variety of diseases using an endothelial ADAS cell produced according to the invention. The skilled artisan would appreciate, based upon the disclosure provided herein, the value and potential of regenerative medicine in treating a wide plethora of diseases including, but not limited to, ischemia, heart disease, including atherosclerotic cardiovascular disease, coronary artery disease, occlusive arterial disease, myocardial ischemia, peripheral vascular occlusive disease, and the like. The present invention encompasses methods for administering endothelial ADAS cells to an animal, including a human, in order to treat a disease where the introduction of new, undamaged cells will provide some form of therapeutic relief.

The skilled artisan will readily understand that endothelial ADAS cells can be administered to an animal whereby upon receiving signals and cues from the surrounding milieu, the cells can further differentiate into mature endothelial cells in vivo dictated by the neighboring cellular milieu. Methods for differentiating ADAS cells to express at least one characteristic of a pre-endothelial cell and/or an endothelial cell in vitro are disclosed herein, and the endothelial ADAS cells can be administered to an animal in the manner described herein. Alternatively, the endothelial ADAS cell can further be differentiated in vitro into a more mature endothelial cell and the mature endothelial cell can be administered to an animal in need thereof.

The endothelial ADAS cell can be prepared for grafting to ensure long term survival in the in vivo environment. For example, cells are propagated in a suitable culture medium for growth and maintenance of the cells and allowed to grow to confluency. The cells are loosened from the culture substrate using, for example, a buffered solution such as phosphate buffered saline (PBS) containing 0.05% trypsin supplemented with 1 mg/ml of glucose; 0.1 mg/ml of MgCl₂, 0.1 mg/ml CaCl₂ (complete PBS) plus 5% serum to inactivate trypsin. The cells can be washed with PBS using centrifugation and are then resuspended in the complete PBS without trypsin and at a selected density for injection.

In addition to PBS, any osmotically balanced solution which is physiologically compatible with the host subject may be used to suspend and inject the donor cells into the host. Formulations of a pharmaceutical composition suitable for parenteral administration comprise the cell combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative.

The invention also encompasses grafting endothelial ADAS cells in combination with other therapeutic procedures to treat disease or trauma in the body, including the CNS, skin, liver, kidney, heart, pancreas, and the like. Thus, endothelial ADAS cells of the invention may be co-grafted with other cells, both genetically modified and non-genetically modified cells which exert beneficial effects on the patient. Therefore the methods disclosed herein can be combined with other therapeutic procedures as would be understood by one skilled in the art once armed with the teachings provided herein.

The endothelial ADAS cells of this invention can be transplanted as endothelial ADAS cells per se into patients using techniques known in the art such as i.e., those described in U.S. Pat. Nos. 5,082,670 and 5,618,531, each incorporated herein by reference, or into any other suitable site in the body.

Transplantation of the cells of the present invention can be accomplished using techniques well known in the art as well as those described herein or as developed in the future. The present invention comprises a method for transplanting, grafting, infusing, or otherwise introducing the cells into a mammal, preferably, a human. Exemplified herein are methods for transplanting the cells into cardiovascular tissue of various mammals, but the present invention is not limited to such anatomical sites or to those mammals. Also, methods that relate to bone transplants are well known in the art and are described for example, in U.S. Pat. No. 4,678,470, pancreatic cell transplants are described in U.S. Pat. No. 6,342,479, and U.S. Pat. No. 5,571,083, teaches methods for transplanting cells to any anatomical location in the body.

The cells may also be encapsulated and used to deliver biologically active molecules, according to known encapsulation technologies, including microencapsulation (see, e.g., U.S. Pat Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 10⁵ to 10⁷ cells. Several macroencapsulation devices may be implanted in the patient. Methods for the macroencapsulation and implantation of cells are well known in the art and are described in, for example, U.S. Pat. No. 6,498,018.

In one aspect of the present invention, ADAS cells are extracted from a donor's adipose tissue and cultured using the methods disclosed herein to administer to a patient in need thereof to elicit a therapeutic benefit to damaged or degenerated myocardium or other cardiovascular tissue in the patient. In addition, the cells which are to be introduced into the individual may be derived from a different donor (allogeneic) or they may be cells obtained from the individual to be treated (autologous). Further, the cells to be introduced into the individual can by obtained from an entirely different species (xenogeneic). In a preferred embodiment the cells are extracted from the adipose tissue of the person into whom they are to be implanted, thereby reducing potential complications associated with antigenic and/or immunogenic responses to the transplant.

The dosage of the endothelial ADAS cells varies within wide limits and may be adjusted to the individual requirements in each particular case. The number of cells used depends on the weight and condition of the recipient, the number and/or frequency of administration, and other variables known to those of skill in the art.

The number of endothelial ADAS cells administered to a patient may be related to, for example, the cell yield after adipose tissue processing. A portion of the total number of cells may be retained for later use or cyropreserved. In addition, the dose delivered depends on the route of delivery of the cells to the patient. Fewer cells may be needed when epicardial or endocardial delivery systems are employed, as these systems and methods can provide the most direct pathway for treating cardiovascular conditions. In one embodiment of the invention, a number of cells to be delivered to the patient is expected to be about 5.5×10⁴ cells. However, this number can be adjusted by orders of magnitude to achieve the desired therapeutic effect.

Between about 10⁵ and about 10¹³ endothelial ADAS cells per 100 kg body weight can be administered to the individual. In some embodiments, between about 1.5×10⁶ and about 1.5×10¹² cells are administered per 100 kg body weight. In some embodiments, between about 1×10⁹ and about 5×10¹¹ cells are administered per 100 kg body weight. In some embodiments, between about 4×10⁹ and about 2×10¹¹ cells are administered per 100 kg body weight. In some embodiments, between about 5×10⁹ cells and about 1×10¹¹ cells are administered per 100 kg body weight.

Endothelial ADAS cells may be administered to a patient in any setting in which myocardial function is compromised. Examples of such settings include, but are not limited to, acute myocardial infarction (heart attack), congestive heart failure (either as therapy or as a bridge to transplant), and supplementation of coronary artery bypass graft surgery. The cells may be extracted in advance and stored in a cryopreserved fashion or they may be extracted at or around the time of defined need. As disclosed herein, the cells may be administered to the patient, or applied directly to the damaged tissue, or in proximity of the damaged tissue, without further processing or following additional procedures to further purify, modify, stimulate, or otherwise change the cells. For example, the cells are cultured in vitro using the methods disclosed herein prior to administering to the patient in need thereof.

The mode of administration of the cells of the invention to the patient may vary depending on several factors including the type of disease being treated, the age of the mammal, whether the cells are differentiated or not, whether the cells have heterologous DNA introduced therein, and the like. The cells may be introduced to the desired site by direct injection, or by any other means used in the art for the introduction of compounds administered to a patient suffering from a cardiovascular disease or disorder.

The endothelial ADAS cells can be administered into a host in a wide variety of ways. Preferred modes of administration are intravascular, intracerebral, parenteral, intraperitoneal, intravenous, epidural, intraspinal, intrastemal, intra-articular, intra-synovial, intrathecal, intra-arterial, intracardiac, or intramuscular. In some embodiments, endothelial ADAS cells are administered to the cardiovascular tissue by direct transplantation. In other embodiments, endothelial ADAS cells are administered to the cardiovascular tissue, i.e., the vascular system, by simple injection.

The endothelial ADAS cells may also be applied with additives to enhance, control, or otherwise direct the intended therapeutic effect. For example, in one embodiment, the cells may be further purified by use of antibody-mediated positive and/or negative cell selection to enrich the cell population to increase efficacy, reduce morbidity, or to facilitate ease of the procedure. Similarly, cells may be applied with a biocompatible matrix which facilitates in vivo tissue engineering by supporting and/or directing the fate of the implanted cells.

Prior to the administration of the endothelial ADAS cells into a patient, the cells may be stably or transiently transfected or transduced with a nucleic acid of interest using a plasmid, viral or alternative vector strategy. The cells may be administered following genetic manipulation such that they express gene products that intended to promote the therapeutic response(s) provided by the cells. Examples of manipulations include manipulations to control (increase or decrease) expression of factors promoting angiogenesis or vasculogenesis (i.e. VEGF), expression of developmental genes promoting differentiation into a specific cell lineage (i.e. MyoD) or that stimulate cell growth and proliferation (i.e. bFGF-1).

The endothelial ADAS cells may also be subjected to cell culture on a scaffold material prior to being implanted. Thus, tissue engineered valves, ventricular patches, pericardium, blood vessels, and other structures could be synthesized on natural or synthetic matrices or scaffolds using the cells prior to insertion or implantation into the recipient.

The cells of the present invention can also be administered in combination with an angiogenic factor to induce or promote new capillary or vessel formation in a subject. The ADAS expressing at least one characteristic of a pre-endothelial cell and/or an endothelial cell can be administered prior to, concurrent with, or following injection of an angiogenic factor. In addition, the cells of the invention may be administered immediately adjacent to, at the same site, or remotely from the site of administration of the angiogenic factor.

In addition, the cells of the invention can be used, for example, to screen in vitro for the efficacy and/or cytotoxicity of compounds, allergens, growth/regulatory factors, pharmaceutical compounds, and the like on pre-endothelial cells and/or endothelial cells, to elucidate the mechanism of certain diseases by determining changes in the biological activity of the cells (e.g., proliferative capacity, adhesion, production of angiogenic factors), to study the mechanism by which drugs and/or growth factors operate to modulate endothelial cell biological activity, to diagnose and monitor diseases in a patient, for gene therapy, gene delivery or protein delivery, and to produce biologically active products. The effect of growth/regulatory factors on the pre-endothelial cell and/or the endothelial cell can be assessed by analyzing the number of living cells in vitro, e.g., by total cell counts, and differential cell counts. This can be accomplished using standard cytological and/or histological techniques, including the use of immunocytochemical techniques employing antibodies that define type-specific cellular antigens. The effect of various drugs on the cells of the invention can be assessed either in a suspension culture or in a three-dimensional system.

The cells of the invention also can be used in the isolation and evaluation of factors associated with the differentiation and maturation of endothelial cells. Thus, the ADAS cells expressing at least one characteristic of a pre-endothelial cell may be used in assays to determine the activity of media, such as conditioned media, evaluate fluids for cell growth activity, involvement with dedication of particular lineages, or the like. Various systems are applicable and can be designed to induce differentiation of the pre-endothelial cells based upon various physiological stresses.

The use of endothelial ADAS cells for the treatment of a disease, disorder, or a condition that affects the cardiovascular system provides an additional advantage in that the endothelial ADAS cells can be introduced into a recipient without the requirement of an immunosuppressive agent. Successful transplantation of a cell is believed to require the permanent engraftment of the donor cell without inducing a graft rejection immune response generated by the recipient. Typically, in order to prevent a host rejection response, nonspecific immunosuppressive agents such as cyclosporine, methotrexate, steroids and FK506 are used. These agents are administered on a daily basis and if administration is stopped, graft rejection usually results. However, an undesirable consequence in using nonspecific immunosuppressive agents is that they function by suppressing all aspects of the immune response (general immune suppression), thereby greatly increasing a recipient's susceptibility to infection and other diseases.

The present invention provides a method of treating a disease, disorder, or a condition that affects the cardiovascular system by introducing endothelial ADAS cells into the recipient without the requirement of immunosuppressive agents. The present invention includes the administration of an allogeneic or a xenogeneic endothelial ADAS cell, or otherwise an endothelial ADAS cell that is genetically disparate from the recipient, into a recipient to provide a benefit to the recipient. The present invention provides a method of using endothelial ADAS cells to treat a disease, disorder or condition without the requirement of using immunosuppressive agents when administering endothelial ADAS cells to a recipient. There is therefore a reduced susceptibility for the recipient of the transplanted endothelial ADAS cell to incur infection and other diseases, including cancer relating conditions that is associated with immunosuppression therapy.

Genetic Modification

The cells of the present invention can also be used to express a foreign protein or molecule for a therapeutic purpose or for a method of tracking their integration and differentiation in a patient's tissue. Thus, the invention encompasses expression vectors and methods for the introduction of exogenous DNA into the cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The isolated nucleic acid can encode a molecule used to track the migration, integration, and survival of the cells once they are placed in the patient, or they can be used to express a protein that is mutated, deficient, or otherwise dysfunctional in the patient. Proteins for tracking can include, but are not limited to green fluorescent protein (GFP), any of the other fluorescent proteins (e.g., enhanced green, cyan, yellow, blue and red fluorescent proteins; Clontech, Palo Alto, Calif.), or other tag proteins (e.g., LacZ, FLAG-tag, Myc, His₆, and the like) disclosed elsewhere herein. Alternatively, the isolated nucleic acid introduced into the cells can include, but are not limited to CFTR, hexosaminidase, and other gene-therapy strategies well known in the art or to be developed in the future.

Tracking the migration, differentiation and integration of the cells of the present invention is not limited to using detectable molecules expressed from a vector or virus. The migration, integration, and differentiation of a cell can be determined using a series of probes that would allow localization of transplanted endothelial ADAS cell. Such probes include those for human-specific Alu, which is an abundant transposable element present in about 1 in every 5000 base pairs, thus enabling the skilled artisan to track the progress of the transplanted cell. Tracking transplanted cell may further be accomplished by using antibodies or nucleic acid probes for cell-specific markers detailed elsewhere herein, such as, but not limited to, CD34, CD31, CD40, CD63, and the like.

The invention also includes an endothelial ADAS cell which, when an isolated nucleic acid is introduced therein, and the protein encoded by the desired nucleic acid is expressed therefrom, where it was not previously present or expressed in the cell or where it is now expressed at a level or under circumstances different than that before the isolated nucleic acid was introduced, a benefit is obtained. Such a benefit may include the fact that there has been provided a system wherein the expression of the desired nucleic acid can be studied in vitro in the laboratory or in a mammal in which the cell resides, a system wherein cells comprising the introduced nucleic acid can be used as research, diagnostic and therapeutic tools, and a system wherein mammalian models are generated which are useful for the development of new diagnostic and therapeutic tools for selected disease states in a mammal.

A cell expressing a desired isolated nucleic acid can be used to provide the product of the isolated nucleic acid to another cell, tissue, or whole mammal where a higher level of the gene product can be useful to treat or alleviate a disease, disorder or condition associated with abnormal expression, and/or activity. Therefore, the invention includes an endothelial ADAS cell expressing a desired isolated nucleic acid where increasing expression, protein level, and/or activity of the desired protein can be useful to treat or alleviate a disease, disorder or condition involving vasculogenesis and/or angiogenesis.

The endothelial ADAS cells cell can be genetically engineered to express an angiogenic factor, for example VEGF, prior to the administration of the engineered ADAS cell into the recipient. The engineered ADAS cell expresses and secretes VEGF at a larger amount compared with an ADAS cell that has not been genetically modified to express such a factor. A benefit of using a genetically modified endothelial ADAS cells in the treatment of a disease, disorder, or a condition that affects vasculogenesis and/or angiogenesis is to increase the therapeutic effects of having endothelial ADAS cells present in the recipient. The increased therapeutic effect is attributed to the increased secretion of VEGF from the engineered endothelial ADAS cells. With the increased secretion of VEGF from the engineered endothelial ADAS cells, a larger amount of VEGF is present for neighboring cells or distal cells to benefit from the VEGF. In addition, the increased amount of VEGF present in the recipient allows a decrease in the time frame during which a patient receives treatment.

It should be understood that the methods described herein may be carried out in a number of ways and with various modifications and permutations thereof that are well known in the art. It may also be appreciated that any theories set forth as to modes of action or interactions between cell types should not be construed as limiting this invention in any manner, but are presented such that the methods of the invention can be more fully understood.

The following examples further illustrate aspects of the present invention. However, they are in no way a limitation of the teachings or disclosure of the present invention as set forth herein.

### EXAMPLES

### Example 1

### Establishment of Primary ADAS Cultures

The stromal vascular fraction (SVF) from white adipose tissue obtained by lipoaspiration was digested in Krebs-Ringer Bicarbonate buffer containing 0.5% BSA and 125 μg/mL collagenase type I (final concentrations) at 37° C. for 80 minutes with vigorous shaking at 10 minute intervals. Following the digestion, the suspension was centrifuged at 1,200 rpm for five minutes at room temperature, shaken vigorously and then centrifuged again at 1,200 rpm for five minutes at room temperature. The lipid/adipocyte layer was aspirated and discarded without disturbing the SVF pellet. The pellet was resuspended/washed in stromal cell medium (DMEM/F12 1:1, 10% FBS, 1×antibiotic/antimycotic) and resuspended in a total volume of 40 mL stromal cell medium. 10 mL of this suspension was added to T-225 flasks containing 40 mL of stromal medium. Non-adherent cells were washed off one to three days following plating and medium was replaced with stromal cell medium supplemented with 5 ng/mL human EGF in the absence of antibiotic/antimycotic. This medium was replaced every three to four days. Confluent cultures were harvested and cryopreserved 14 days following plating. These cells were designated as ADAS passage 0 (P0).

### Example 2

### Treatment of ADAS Cells

In two separate experiments, ADAS (P0) cells from a single donor were plated in T-83 flasks at a density of about 6×10³ cells/cm² (passage 1). Control ADAS cells were cultured in expansion medium comprising DMEM/F12 (1:1), 10% FBS, 5 ng/mL hEGF and 1 ng/mL hFGF with media changes every two to four days. ADAS cells in the treatment group were subjected to a stepwise treatment regimen beginning with six days in MII medium (DMEM/F12, N2 supplement, B27 supplement, 2.3 mM glutamine, 10 ng/mL hFGF) with medium changes on days 1, 3 and 5 following plating. On day 7, the cultures were rinsed twice with D-PBS and then the ADAS cells were incubated for four days in MIII medium (DMEM/F12, N2 supplement (Invitrogen, Carlsbad, Calif.), B27 supplement (Invitrogen, Carlsbad, Calif.), 2.3 mM glutamine, 10 mM nicotinamide, 2% FBS). MIII was replaced on day 10 and both control and treated ADAS cells were harvested via trypsinization on day 11 and subjected to flow cytometry.

### Flow Cytometry

Flow cytometry was performed on control and MII/MIII treated cells for phenotypic characterization and to identify potential cellular responses to the MII/MIII medium treatment regimen. For conjugated monoclonals, the cells were washed once in 1 mL flow wash buffer (1×DPBS, 0.5% BSA and 0.1% sodium azide) and centrifuged at about 6000×g for 20 seconds. Cells were suspended in 1.3 mL of blocking buffer (wash buffer with 25 μg/mL mouse Ig), incubated on ice for ten minutes, and then aliquoted into 100 μL aliquots. Appropriate monoclonal antibodies were added to their respective aliquots. The appropriate isotype control combinations were included to correspond to the monoclonal isotype combinations used in the experiment. Three monoclonal antibodies were included per tube. The concentrations of antibody used in the experiment were the vendor recommended concentrations. Antibodies used to phenotype the ADAS cells include (all antibodies were from BD-Pharmingen, San Jose, Calif. unless otherwise indicated): CD80 (Caltag, Burlingame, Calif.), CD86 (Caltag), CD14; CD45, CD34, CD133 (Miltenyi Biotech, Auburn, Calif.); CD90, CD105 (Caltag), HLA-DR; CD63, CD166, MHC Class I; CD44 (Cell Sciences, Canton, Mass.), CD73, CD54; CD31, CD13, CD40; CD29 (Caltag), CD49a, CD11a. All tubes were incubated on ice and protected from light for 30 minutes. The cells were washed once in 2 mL wash buffer (about 650×g for five minutes) and then fixed in 200 μL of 1% paraformaldehyde.

For unconjugated monoclonal antibodies, the ADAS cells were harvested, washed and blocked as described elsewhere herein. Primary antibodies (VEGFR2 [KDR] and von Willebrand Factor) were added (10 μg/mL) and the cells were incubated for about 30 minutes on ice. The cells were washed once in 2 mL wash buffer (650×g for five minutes) and resuspended in 100 μL wash buffer. Goat anti-mouse PE conjugated secondary antibody at a concentration of 0.5 μg/mL was added to the suspensions containing primary antibody as well as a “secondary antibody only” control and the cells were incubated on ice and protected from light for 15 minutes. A final wash in 2 mL of flow wash buffer (650×g for five minutes) was performed and the cells were fixed as described above.

10,000 events (cells) were acquired per antibody set on a Becton Dickinson FACSCaliber flow cytometer using CELLQuest acquisition software (Becton Dickinson, Franklin Lakes, N.J.) and the data was analyzed with Flow Jo analysis software (Tree Star, Ashland, Oreg.).

### Morphology

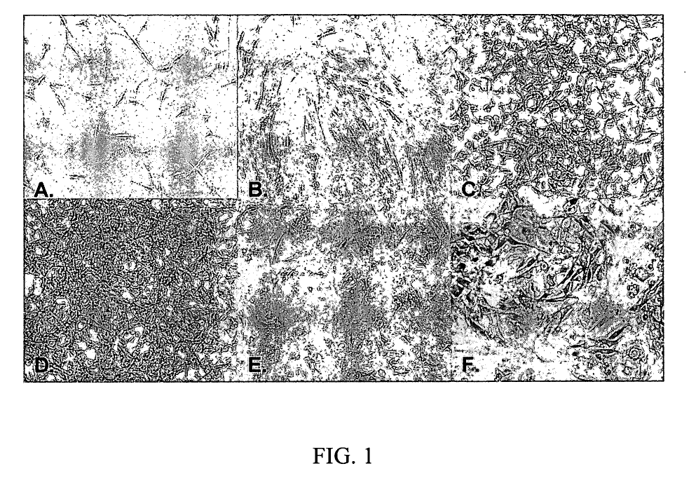

FIG. 1 shows photomicrographs representative of the untreated and MII/MIII treated ADAS cultures. A spindle-shaped, fibroblast-like morphology was observed in pre-treated and untreated control cultures (FIG. 1A and FIG. 1B) while a morphological change was observed in MII treated cultures four days following the initiation of treatment (FIG. 1C and FIG. 1D). MII treated cells were less fibroblastic in appearance with a considerable number of round, phase bright cells both adherent and non-adherent. Furthermore, cells at this stage appeared to form a “network” of cell to cell connections. A further treatment of the cells with MIII again changed the overall morphology of the culture yielding a heterogeneous mixture of cells resembling those seen during the MII treatment in addition to cells forming “cobblestone” type areas resembling cultured endothelial cells (FIG. 1E and FIG. 1F).

### Phenotypic Characterization

Control and MII/MIII treated ADAS cells were phenotypically characterized for surface marker expression using antibodies directed against various molecules typically expressed by cells of stromal, hematopoietic, or endothelial lineages. Table 1 shows the results of this characterization (values are percent positively staining cells for the listed surface marker). Following 11 days in culture untreated passage 1 ADAS cells expressed CD13, CD29, CD31, CD40, CD44, CD49a, CD54, CD63, CD73, CD80, CD90, CD105, CD133, CD166, MHC Class I, and VEGF receptor 2 (flk-1). When treated with the MII/MIII regimen, significant changes (>20%) were noted in the average percentage of cells expressing surface markers CD31 (+56.5%), CD34 (+67.9%), CD40 (+27.4%), CD63 (+38.0%), CD105 (−25.3%), and CD166 (−36.1%).

TABLE 1
Phenotypic characterization of control and MII/MIII treated ADAS cells

| | Experiment 1 | | Experiment 2 | |
|---|---|---|---|---|
| Antigen | Untreated | Treated | Untreated | Treated |
| CD11a | <0.01 | <0.01 | ND | ND |
| CD13 | 98.3 | 98.2 | 98.1 | 96.8 |
| CD14 | <0.01 | <0.01 | <0.01 | <0.01 |
| CD29 | 97.9 | 97.9 | 97.6 | 93 |
| CD31 | 25.4 | 90 | 22.9 | 71.2 |
| CD34 | 0.8 | 64.2 | 1.6 | 74 |
| CD40 | 46.9 | 80.3 | 60 | 81.3 |
| CD44 | 98.4 | 98.2 | 98.1 | 96.8 |
| CD45 | <0.01 | <0.01 | <0.01 | <0.01 |
| CD49a | 4.6 | 1.2 | 12.8 | 10.8 |
| CD54 | 75.2 | 77.1 | 71.8 | 78.1 |
| CD63 | 45.9 | 97.1 | 66.5 | 91.3 |
| CD73 | 98.2 | 97.6 | 97.9 | 96.4 |
| CD80 | 4.4 | 5.4 | 10.8 | 18.7 |
| CD86 | <0.01 | <0.01 | <0.01 | <0.01 |
| CD90 | 98 | 98.3 | 98.2 | 97.1 |
| CD105 | 23.7 | <0.01 | 44.9 | 18.1 |
| CD133 | 9.6 | 3.4 | 4.1 | 6.3 |
| CD166 | 49.9 | 19.9 | 61.1 | 18.9 |
| MHC Class I | 96.8 | 97.9 | 88.6 | 89 |
| MHC Class II | 0.1 | 1.5 | 1.4 | 1.1 |
| VEGF-r 2 | ND | ND | 44.9 | 30.8 |
| Von Willebrand factor | ND | ND | 2.4 | 2.3 |

* BG = Background

The disclosure herein demonstrates a novel method of using a media treatment scheme to treat undifferentiated ADAS cells to arrive at a population of endothelial ADAS cells. Although the phenotypic characterization of the cells presented herein does not include all potential endothelial cell surface markers, it was observed that the cell population generated by the methods disclosed herein is strongly positive for CD34 (a marker of pre-endothelial cells and hematopoietic stem cells) and CD31 (mature endothelial cell), as well as positive for CD40 and CD63, which is consistent with the phenotypic characterization for commitment towards an endothelial cell type. The data presented herein demonstrated the potential of using the method disclosed herein to generate large numbers of CD34+, CD31+ pre-endothelial cells from easily expanded endothelial ADAS for use clinically. In addition, the present discovery allows for an attractive approach for expanding endothelial ADAS cells in a large scale environment using the treatments as described herein to induce the endothelial cell marker positive phenotype and manufacturing of pre-endothelial cells. Further, the expression of CD34 together with CD40 as well as CD80 (to a lesser extent) on treated ADAS cells suggests that hematopoietic precursors may be also induced.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An isolated adipose tissue-derived adult stromal (ADAS) cell induced to express at least one characteristic of a pre-endothelial cell.

2. The cell of claim 1, wherein said cell is induced to differentiate in vitro.

3. The cell of claim 1, wherein said cell is induced to differentiate in vivo.

4. The cell of claim 1, wherein exogenous genetic material has been introduced into said cell.

5. The cell of claim 1, wherein said cell is derived from a human.

6. The cell of claim 1, wherein said cell expresses at least one of CD34 and CD31.

7. The cell of claim 1, wherein said cell expresses at least one of CD34 and CD31 at a higher level when compared with the expression level of CD34 and CD31, respectively, from an otherwise identical ADAS cell not induced to express at least one characteristic of a pre-endothelial cell.

8. The cell of claim 1, wherein said cell expresses at least one of CD34, CD31, CD40, CD63, or a combination thereof.

9. The cell of claim 1, wherein said cell expresses at least one of CD34, CD31, CD40, CD63, or a combination thereof at a higher level when compared with the expression level of CD34, CD31, CD40 and CD63, respectively, from an otherwise identical ADAS cell not induced to express at least one characteristic of a pre-endothelial cell.

10. A method of differentiating an isolated adipose tissue-derived adult stromal (ADAS) cell to express at least one characteristic of a pre-endothelial cell, the method comprising incubating said cell in MII medium followed by incubating said cell in MIII medium.

11. The method of claim 10, wherein said cell is derived from a human.

12. The method of claim 10, wherein said MII medium comprises N2 supplement, B27 supplement, glutamine and fibroblast growth factor (FGF).

13. The method of claim 12, wherein the concentration of said glutamine is about 2.3 mM.

14. The method of claim 12, wherein the concentration of said FGF is about 10 ng/mL.

15. The method of claim 10, wherein said MIII medium comprises N2 supplement, B27 supplement, glutamine, nicotinamide and fetal bovine serum (FBS).

16. The method of claim 15, wherein the concentration of said glutamine is about 2.3 mM.

17. The method of claim 15, wherein the concentration of said nicotinamide is about 10 mM.

18. The method of claim 15, wherein the concentration of said FBS is about 2%.

19. A differentiation medium for differentiating an isolated adipose tissue-derived adult stromal (ADAS) cell into a cell exhibiting at least one characteristic of a pre-endothelial cell, wherein said medium is supplemented with N2 supplement, B27 supplement, glutamine and fibroblast growth factor (FGF), further wherein said medium is designated as MII medium.

20. The medium of claim 19, wherein the concentration of said glutamine is about 2.3 mM.

21. The medium of claim 19, wherein the concentration of said FGF is about 10 ng/mL.

22. A differentiation medium for differentiating an isolated adipose tissue-derived adult stromal (ADAS) cell into a cell exhibiting at least one characteristic of a pre-endothelial cell, wherein said medium is supplemented with N2 supplement, B27 supplement, glutamine, nicotinamide and fetal bovine serum (FBS), further wherein said medium is designated as MIII medium.

23. The medium of claim 22, wherein the concentration of said glutamine is about 2.3 mM.

24. The medium of claim 22, wherein the concentration of said nicotinamide is about 10 mM.

25. The medium of claim 22, wherein the concentration of said FBS is about 2%.

26. A method of inducing vasculogenesis in an animal comprising:
a) inducing an isolated adipose tissue-derived adult stromal (ADAS) cell to express at least one characteristic of a pre-endothelial cell; and
b) administering said cell so induced into said animal.

27. The method of claim 26, wherein said ADAS cell is isolated from said animal.

28. The method of claim 26, wherein said ADAS cell is isolated from an allogeneic donor.

29. The method of claim 26, wherein said ADAS cell is isolated from a xenogeneic donor.

30. The method of claim 26, wherein said ADAS cell is derived from a human.

31. A method of determining the ability of a compound to affect the differentiation of an isolated adipose tissue-derived adult stromal (ADAS) cell into a pre-endothelial cell and/or endothelial cell, the method comprising:
a) culturing said ADAS cell in a stromal cell medium for a period of time;
b) replacing said stromal cell medium with a differentiation medium comprising a compound or a control vehicle;
c) incubating said ADAS cell in said differentiation medium comprising said compound or said control vehicle for a period of time;
d) determining the number or percentage of differentiated cells using said differentiation medium comprising said compound from step (c);
e) determining the number of percentage of differentiated cells in the cells using said differentiation medium containing said vehicle alone from step (c);
f) comparing the number or percentage of differentiated cells from steps (d) and (e);
g) a greater number of percentage of differentiated cells from step (d) compared to the number of percentage of differentiated cells from step (e) indicates that said compound is capable of inducing differentiation of said ADAS cell into a pre-endothelial cell and/or endothelial cell.

32. The method of claim 31, wherein said cell is derived from a human.
